(19) United States
(12) Patent Application Publication
(10) Pub. No.: US 2002/0041869 A1
(43) Pub. Date: Apr. 11, 2002

(54) Methods for treating cells

(75) Inventors: Selman Abdul-Halim Ali, Sheffield (GB); June Lynam, Derbyshire (GB); Robert Charles Rees, Sheffield (GB); Cornelia Suzanna McLean, Cambridge (GB)

Correspondence Address: KLARQUIST SPARKMAN, LLP, 121 SW SALMON STREET, SUITE 1600, PORTLAND, OR, 97204, US

(73) Assignee: The Nottingham Trent University and Cantab Pharmaceuticals Research Limited

(21) Appl. No.: 09/878,136

(22) Filed: Jun. 6, 2001

(30) Foreign Application Priority Data
Jun. 6, 2000 (GB) 0013817.2

(51) Int. Cl. A61K 48/00; C12N 15/86

(52) U.S. Cl. 424/93.21; 424/93.7; 435/456

(57) ABSTRACT

Methods for treating cells, e.g. proliferating cells such as tumor cells, comprise exposing them to cell-damaging agents (which can cause indirect damage, e.g. as vectors for delivery of a gene encoding a cytokine or antigen) and to antigen-presenting cells so as to damage them or reduce their proliferation.

### FIELD OF THE INVENTION

This invention relates to methods for treating cells, e.g. proliferating cells, for example hyperproliferating cells, such as tumour cells, so as to damage them or reduce their proliferation.

### BACKGROUND OF THE INVENTION AND PRIOR ART

Administration to mammals of cytokines is known as such, but is often poorly tolerated by the host (A Mire-Sluis, TIBTech Vol. 11 (1993); M S Moore, Ann Rev Immunol 9 (1991), 159-191).

It is also known to modify live virus vectors to carry genes encoding a cytokine or tumour antigen, see e.g. WO 96/26267 (Cantab Pharmaceuticals: Inglis et al) and references cited therein including WO 94/16716 (Virogenetics Corporation: Paoletti et al.).

Gene sequences of a large number of cytokine genes and other immunomodulatory proteins are known. For example, human GM-CSF and its gene are described in M Cantrell et al., Proc. Nat. Acad. Sci. 82 (1985), pp 6250-6254; F Lee et al., Proc. Nat. Acad. Sci. 82 (1985), pp 4360-4364 and G Wong et al., Science 228 (1985), pp 810-815. The gene encoding murine GM-CSF is also known (Gough et al., EMBO Journal 4, pp 645-653). Other known genes include for example, the gene encoding the human RANTES lymphokine (T J Schall et al., 1988, J. Immunol., 141 (3), pp 1018-1025) and the gene encoding human lymphotactin (J Kennedy et al., J. Immunol., July 1995, 266, pp 1395-1399) and also murine lymphotactin (G S Kelner et al., Science, November 1994, 155 (1), pp 203-209).

Virus vectors which are defective in respect of a gene essential for production of infectious virus, such that the virus can infect normal host cells and undergo replication and expression of viral antigen genes in such cells but cannot produce infectious virus are known and described in specification WO 92/05263 (Immunology Limited: Inglis et al) and WO 94/21807 (Cantab Pharmaceuticals Research Limited: Inglis et al).

WO 92/05263 (Immunology Limited: Inglis et al) particularly describes an HSV virus which is disabled by functional deletion of a gene encoding the essential glycoprotein (gH) which is required for virus infectivity.

It is also known to administer dendritic cell preparations to mammals. BM Colombo et al. (Immunology, 2000, 99 (1): 8-15) describes vaccination of mice with tumour extract-loaded dendritic cells and subsequent generation of a CD-4 antigen specific cell-mediated cytotoxic protective immune response.

It is also known that certain bacteria can possess anti-tumour activity. H Akaza et al.(Cancer, 1993, 72 (2): 558-563) describes the anti-tumour effects of Bacillus Calmette-Guerin (BCG) against urinary bladder cancer.

### SUMMARY AND DESCRIPTION OF THE INVENTION

According to an aspect of the invention there is provided a combination treatment for treating target cells, for example proliferating cells, e.g. hyperproliferating cells, e.g. tumour cells. The treatment comprises the steps of:

a) exposing target cells to a cell-damaging agent, for example, an agent which is capable of producing cell inflammation and/or lysis, and then

b) exposing said target cells to a preparation of antigen-presenting cells, e.g. dendritic cells. The cell-damaging agent can contact the target cells, or can enter, or be taken up by them. This can enable the antigen-presenting cells to encounter substances such as antigens, that can be produced by exposure of the target cells to the cell-damaging agent in step a) of the process.

In alternative embodiments, step b) can precede step a) or be carried out at the same time as step a).

The invention provides in one aspect a method of treating target cells to damage them and/or reduce their proliferation: the method comprises the steps of (a) exposing the target cells to a cell-damaging agent, and also (b) exposing said target cells to a preparation of antigen-presenting cells, thereby to damage said cells and/or reduce their proliferation. Step (b) can be carried out after step (a), e.g. at least about 30 minutes after step (a). The antigen-presenting cells can consist essentially of dendritic cells. The cell-damaging agent can consist essentially of a vector, e.g. a virus vector, for gene delivery. The damage produced by the agent can be indirect, e.g. by the immune response generated. Such a virus vector can comprise one or more gene sequences encoding an immunomodulatory protein and/or a tumour antigen, or a functional fragment thereof. The cell-damaging agent and the antigen-presenting cells can be delivered to target cells in vivo, or alternatively to in-vitro cells, in which case the treated target cells can then be implanted or administered into a subject.

Thus in one form of embodiment, a method of treating cell proliferation in a subject can comprise administering to said subject separately or concurrently a preparation (a) which consists essentially of a cell-damaging agent, and a preparation (b) which consists essentially of an antigen-presenting cell preparation, in combination with a pharmaceutical excipient.

The invention also provides a combined preparation for use in therapy to damage cells and/or reduce their proliferation, said preparation comprising (a) a cell-damaging agent and (b) a preparation of antigen-presenting cells, wherein said components (a) and (b) are arranged for sequential or simultaneous use.

The invention also extends to uses of a cell-damaging agent and a preparation of antigen-presenting cells in the manufacture of a medicament for their sequential or simultaneous use to treat target cells to damage them and/or reduce their proliferation.

A pharmaceutical according to one form of the invention can comprise a cell-damaging agent and a preparation of antigen-presenting cells in combination with a pharmaceutically acceptable excipient (e.g. as separate physical compositions for sequential use as described herein). Pharmaceutically acceptable excipients can be used in per-se known manner.

Further aspects of the invention are: a cell preparation obtainable by treating target cells in vitro as described herein, and a method of treating cell proliferation in a subject which comprises administering to said subject such a cell preparation and also use of a cell preparation obtainable by treating target cells in vitro as described herein in the manufacture of a medicament to damage cells and/or reduce their proliferation.

In a preferred embodiment, treating target cells according to a method of the invention can reduce proliferation of said treated target cells and/or can kill the target cells and/or evoke an immune response, e.g. a local immune response, e.g. a local anti-tumour response, against the treated cells.

Examples of proliferating cells which can be treated by the process of the invention are cells present in a cell mass, e.g. malignant tumour cells, e.g. in breast, melanoma, hepatic, and head or neck tumours. Alternatively, the proliferating cells can be non-malignant cells of for example benign tumours such as genital warts; or for example proliferating endometrial cells.

Examples of cell-damaging agents which can be used in the process of the invention include for example micro-organisms, e.g. preparations of virus and/or bacteria, e.g. immunogenic preparations such as those suitable for vaccine use; radiation, e.g. in doses appropriate for selective cell killing; substances such as anti-tumour pharmaceuticals, e.g. cisplatin; or other chemotherapeutic agents, e.g. doxorubicin, etoposide, or paclitaxel. Cell damaging agents can include for example agents which can cause indirect damage to cells, e.g. as a result of the immune response generated.

In a preferred embodiment of the invention the cell-damaging agent can be a micro-organism, for example a virus or a bacterium. The micro-organism can be a live organism (e.g. a genetically attenuated organism) or a killed preparation.

Antigen-presenting cells which can be used in the process of the invention can for example for example dendritic cells or macrophages.

Antigen-presenting cells are often migratory cells and it is believed that this property can depend inter alia upon age and maturity of the cells.

When the antigen-presenting cells are cells having substantial migratory ability, it can be particularly useful to carry out step b) of the process at a time period after step a) which is at least about 30 minutes, e.g. at least about 1 hour, or about 2 hours or more, e.g. 3 hours, 4 hours, 5 hours or more.

Antigen-presenting cells which have substantial migratory activity, e.g. dendritic cells, can be mature or pre-mature cells at least about seven days old, e.g. at least about eight or nine days old. In embodiments mentioned below the cells can for example be about seven days old.

Alternatively, and for example when the antigen-presenting cells lack substantial migratory ability, it can be particularly useful to carry out step b) of the process after step a) of the process of the invention. On the other hand, the steps can also be carried out with step b) first, or at the same time as step a).

Antigen-presenting cells which lack substantial migratory activity, e.g. dendritic cells, are normally, for example, immature cells which are less than about seven days old, e.g. cells which are less than about six days old, e.g. about five days old. Cells lacking substantial migratory activity can for example be dendritic cells displaying levels of dendritic cells markers, e.g. CD40, CD11, CD80, and CD45 markers, lower than the levels found in corresponding mature dendritic cells.

Migratory activity of a preparation of antigen-presenting cells, e.g. dendritic cells, can be determined using standard techniques known in the art, for example, using a Boyden chamber migration assay (E Ortega et al., Mol Cell Biochem, January 2000, 203 (12), pp 113-7; S Dunzendorfer et al., Immunol. Lett., 2000, 71 (1), pp 5-11) or in an in vivo model by injecting labeled dendritic cells (MB Lappin et al., Immunology, 1999, 98 (2), pp 181-188). Migratory activities can range from those shown by mature dendritic cells down to lack of migration.

In another embodiment of the invention the target cells treated by the process of the invention, e.g. proliferating cells, can then be further contacted with another cell-damaging agent, e.g. an agent capable of causing inflammation and/or lysis, following exposure of the target cells to antigen-presenting cells, e.g. dendritic cells. This second cell-damaging agent preparation can be another dose of the same agent, or else a dose of a different agent, compared to that used in step a).

It can be especially desirable to administer this second cell-damaging agent at lest about 1 day after the antigen-presenting cells, e.g. about 2 or 3 days after, e.g. up to about 5 or 6 days after.

Examples of viruses which are capable of producing cell inflammation and/or causing lysis and which can usefully be used in step a) of the process of the invention include for example: herpesvirus or adenovirus. Mutant herpesviruses can for example be based on HSV1, or HSV2. It is considered that both a killed and a live virus often produce inflammation, whilst cell lysis is associated with a live virus, e.g. a genetically-defective but infective virus.

Particularly useful examples of viruses which can be used in a method according to the invention are live viruses, e.g. live virus vectors, e.g. live defective virus vectors, e.g. genetically disabled virus vectors, e.g. a mutant virus whose genome is defective in respect of a gene essential for the production of infectious virus such that the virus can infect cells and undergo replication and expression of viral antigen genes but cannot produce infectious virus. Examples of such defective herpesviruses, e.g. herpesvirus vectors and of methods of producing them are described in specifications: WO 92/05263 (Immunology Limited: Inglis et al.), WO 96/26267 (Cantab Pharmaceuticals Research Limited: Inglis et al.) and WO 96/04395 (Lynxvale Limited: P Speck) and documents cited therein.

Examples of bacteria which can usefully be used as cell-damaging agents in the process of the invention include for example, lactic acid bacteria, bacillus Calmette-Guerin (BCG), and also for example bacteria which can enter cells.

When the cell-damaging agent is a micro-organism it can comprise heterologous nucleotide sequences, e.g. one or more gene sequences encoding one or more immunomodulatory proteins and/or one or more tumour antigens, or functional fragments thereof.

Immunomodulatory proteins which can usefully be delivered to cells in the present connection include cytokines and other immune system proteins that can enhance the host immune response, for example proteins such as those mentioned in specification WO 96/26267 (Cantab Pharmaceuticals Research Limited: Inglis et al.), the contents of which is incorporated herein by reference. Examples of such immunomodulatory proteins which can be particularly useful in the context of the present invention include: granulocyte-macrophage colony stimulating factor (GM-CSF), RANTES, OX40, OX40L, CD40, CD40L, interleukins (e.g. IL-2, IL-12) and tumour necrosis factor alpha. The protein can be other than tumour necrosis factor alpha (TNF-alpha).

In certain embodiments the micro-organism, e.g. virus, need not contain additional heterologous DNA, in particular it need not carry a gene encoding a cytokine. In certain embodiments the virus can thus for example be free of any one or all of the following cytokine genes: granulocyte-macrophage colony stimulating factor (GM-CSF), RANTES, OX40, OX40L, CD40, CD40L, interleukins (e.g. IL-2, IL-12), and tumour necrosis factor alpha.

A preparation of antigen-presenting cells can be prepared in a number of ways as is known in the art. A preparation of antigen-presenting cells, e.g. dendritic cells as used herein can be tested when desired for activity, i.e. for its antigen-presenting activity and ability to activate T-cells, for example by mixing antigen-presenting cells with T-cells and then determining T-cell activation using standard methods.

When it is desired to use a dendritic cell preparation as active antigen-presenting cell preparation, it can be isolated from any of a number of sources, e.g. from blood, bone marrow or spleen.

Active dendritic cells can be isolated from bone marrow in known manner, e.g. using the method of Inaba et al., J. Exp. Med. 1992, 176 (6), pp 1693-1702, or an adaptation of the method of Inaba et al. e.g. as further described herein.

It can be desired to obtain the dendritic cell preparation from blood, e.g. from fresh human blood, for example by using the method of M. Thurnher et al. Exp. Hematology 1997, 25, pp 232-237, or an adaptation thereof, e.g. use of a lower final concentration of IL-4 such as 500 U/ml, addition of cytokines on day 0 and day 6, and/or addition of TNF for 2 days only. Also, the concentration of dendritic cells can be lowered by dilution on day 3 as described by Thurnher et al., or alternatively if fully mature dendritic cells are required it can be desirable to dilute after that, e.g. at day 6. When the human blood used is frozen instead of fresh it can be desired to eliminate the second adherence step of Thurnher et al. on day 1.

It can also be desirable to use autologous dendritic cells isolated from blood for the purpose of carrying out examples of the invention, i.e. dendritic cells isolated from the blood of a subject to be treated according to the invention. This can ensure that the treated subject does not mount an immune response against the dendritic cell preparation when it is administered to said subject.

These methods can yield active cell preparations substantially free of cells able to suppress antigen-presenting activity.

The antigen-presenting cells, e.g. dendritic cells, can be grown in a medium comprising GM-CSF. Under these conditions among others, the micro-organism can carry a gene encoding GM-CSF, e.g. human GM-CSF, as this can produce particularly useful results.

When the micro-organism encodes one or more tumour antigens, the tumour antigen(s) can be one or more of those expressed by the target cells. Examples of such tumour antigens include: gp100 antigen expressed by melanoma cells (Y Kawakami et al. PNAS, July 1994), and prostate specific antigen (PSA) expressed by prostate tumours (P Schulz e al., Nucleic Acids Research, Vol 17 (10), pp3981).

In one way of carrying out the invention, both the cell-damaging agent and the antigen-presenting cells, e.g. dendritic cells, can be delivered to target cells in vivo, for example by direct injection into a tumour cell mass.

Alternatively, the cell-damaging agent can be delivered to target cells ex-vivo, e.g. to isolated target cell preparations, for example to autologous tumour cells, or to heterologous tumour cells. After delivery of the cell-damaging agent ex-vivo, the treated target cells, e.g. cells in contact with the agent or containing the agent, can then be implanted into a subject which it is desired to treat (this part of the procedure can for example be as described by SA Ali et al., Cancer Research 2000, 60, pp 1663-1670), e.g. by injection, e.g. by injection sub-cutaneously, e.g. by direct intra-tumoural injection. This can be followed by delivery of antigen-presenting cells, e.g. dendritic cells, to said subject, such that after delivery the antigen-presenting cells can come into contact with said treated target cells. To facilitate this the antigen-presenting cells can be delivered to a subject by injection at or near the site of delivery of the ex-vivo virus treated cells, e.g. by injection sub-cutaneously.

In methods according to the invention, antigen-presenting cells can be delivered in vivo, particularly antigen-presenting cells with substantial migratory activity, e.g. mature or pre-mature antigen-presenting cells, at an interval following exposure of target cells to a cell-damaging agent ex-vivo. This interval can be for example at least about 30 minutes, e.g. at least about 1, 2, 4, 8, 12 or 24 hours, or an interval of up to any of those periods named.

In yet a further aspect of the invention, the cell-damaging agent can be delivered to target cells ex-vivo, this then be followed by ex-vivo delivery to the treated target cells of an antigen-presenting cell preparation, e.g. dendritic cell preparation. It can be particularly useful to deliver the antigen-presenting cells, e.g. cells with substantial migratory activity, such as mature or pre-mature antigen-presenting cells, after an interval following exposure of target cells to the cell-damaging agent ex-vivo, which is as described above. The preparation comprising treated target cells and dendritic cells can then be administered to a subject of treatment such as a human, e.g. by direct injection.

Delivery of a cell-damaging agent, and/or antigen-presenting cells ex-vivo as described above, can be followed by administration of treated cells to a subject, as described above, especially for example when the target cells in vivo, e.g. target tumour cells, are relatively inaccessible by direct injection. Alternatively, when the target cells are relatively inaccessible, the mutant virus and/or dendritic cells can be delivered in vivo to said target cells, e.g. by using endoscopic delivery methods.

In yet a further aspect of the invention, antigen-presenting cells, particularly cells with substantial migratory activity, can be delivered to a patient by sub-cutaneous injection so as to facilitate their accumulation in the T cell areas of lymph nodes (A A O Eggert et al., Cancer Research 1999, Vol 59, No. 14, pp 3340-3345).

Preparations comprising treated target cells, e.g. target cells in contact with and/or containing a) a cell-damaging agent, and/or b) antigen-presenting cells, e.g. dendritic cells, in combination with a pharmaceutically acceptable excipient are also provided by the invention. Such preparations can be formulated by readily adapting known methods for therapeutically useful compounds. Suitable vehicles and their formulation are described in Remingtons Pharmaceutical Science by E. W. Martin (Mack Publishing Company, 1990). The compositions can contain minor amounts of auxiliary substances such as stabilisers and/or pH buffering agents.

When the cell-damaging agent is a herpesvirus it can be useful to deliver to the target cells from about 1×10ˆ 3 to about 1×10ˆ 8 pfu of virus, e.g. from about 1×10ˆ 4 to about 1×10ˆ 7 pfu. When the cell-damaging agent is an adenovirus it can be useful to deliver to the cells from about 1×10ˆ 3 to about 1×10ˆ 3 pfu of virus. It can be also useful to deliver at least about 2×10ˆ 5 dendritic cells, e.g. at least about 3×10ˆ 5, 4×10ˆ 5, or 5×10ˆ 5 or higher amounts of dendritic cells. A subject of the process can receive multiple treatments.

When the cell-damaging agent is an anti-tumour pharmaceutical, for example a chemotherapeutic drug, it can be useful to deliver standard dosages of chemotherapeutic drugs. For example, for doxorubicin, dosage is usually calculated on the basis of body area, and doses which can usefully be administered as part of the method of the invention are 60-70 mg per sq.m., e.g. 30-40 mg per sq.m. This can be administered as a single dose, by for example intravenous administration, e.g. every three weeks.

An example of a DNA-damaging agent which can be used to treat proliferating cells in examples of methods of the invention is cisplatin. This can be given by infusion over a period of hours, e.g. in doses upwards of 20 mg per sq.m. (body area of a subject to be treated), e.g. 60-70 mg per sq.m., e.g. 30-40 mg per sq.m., administered, for example, every three weeks.

In certain preferred examples of the invention, treatment of a target tumour comprises administering to the tumour a genetically disabled mutant herpesvirus encoding a gene expressing the cytokine GM-CSF, followed by contacting said tumour with a preparation of dendritic cells, and optionally then by further contacting said target tumour cells with a mutant herpesvirus encoding GM-CSF.

### EXAMPLES

Examples of the invention are described below without intent to limit its scope.

First, the description below is of materials suitable for carrying out the several examples of the treatment process provided by the invention, that are then described.

A genetically disabled mutant herpesvirus encoding a gene expressing murine GM-CSF was made as described in specification WO 96/26267 (Cantab Pharmaceuticals Research Limited: Inglis et al.).

A preparation of dendritic cells was made using the following procedure:

Bone marrow was extracted from mice, this was then cultured for seven days in a culture medium comprising GM-CSF and IL-4 (the method used is described in J. Exp. Med. 1992, 176 (6), pp 1693-1702, K Inaba et al.).

Dendritic cells were then obtained from this extracted murine bone marrow using the method as follows (which is adapted from that described in Inaba et al. above):

The hind limbs of a mouse were harvested aseptically and placed in a sterile wash solution (Phosphate buffered saline comprising antibiotics as follows: penicillin 50 International Units/ml, streptomycin 50 micrograms/ml, fungizone 0.25 mg/ml) and left on ice for five minutes. The muscle tissue was then aseptically removed from the bones. The ends of the bones were then removed and the marrow flushed from the bone with serum-free RPMI medium (Gibco Life Sciences, UK) using a 25 g needle and 10 ml syringe. The marrow was collected and placed in a sterile container and the marrow cells were then suspended using gentle agitation. A 10 microliter sample of marrow cell suspension was removed for cell counting using a haemocytometer.

The marrow cell suspension obtained was then centrifuged (1500 rpm, five minutes) and the supernatant removed, and the cell pellet re-suspended in dendritic cell generation medium (RPMI 1640 medium containing supplements as follows: 10 mM Hepes (Gibco Life Sciences, UK), 50 mM mercaptoethanol, 20 mg/ml gentamycin, 50 International Units/ml penicillin, 50 micrograms/ml streptomycin, 0.25 mg/ml fungizone, 20 ng/ml murine GM-CSF, obtained from Pepro TechEC Ltd., London, UK). The cells were re-suspended at a concentration of 10ˆ 6 leukocytes per ml.

Finally, the cells obtained were seeded in 24-well tissue culture plates (at 10ˆ 6 bone marrow cells per ml per well) and incubated at 37 deg C. (in 5% carbon dioxide in air, humidified atmosphere). At days 2 and 4 following cell seeding, the cultured cells were washed with dendritic cell culture medium to remove any non-adherent cells, and the cells further incubated in fresh culture medium. At day 7 following cell seeding, cultured dendritic cells were harvested as follows: clusters of loosely adherent cells were gently dislodged by washing the culture wells with culture medium, the cells collected were pooled and then centrifuged (1500 rpm, 3 minutes), and the dendritic cell pellet obtained was re-suspended in serum-free RPMI medium and stored on ice until required.

A virus expressing murine GM-CSF, prepared as described above, followed by administration of a preparation of dendritic cells, prepared as described above, was used to treat tumours in vivo in a mouse model as follows:

### Example 1

In this example of a treatment process according to the invention a mouse model system was used to test the efficacy of administration of a virus expressing murine GM-CSF followed by administration of a preparation of dendritic cells to treat tumours.

Mice expressing the colorectal tumour cell line CT26 were obtained as described in S Todryk et al., 1999, Human Gene Therapy, 10, pp 2757-2768.

Groups of mice expressing CT26 tumours were treated by intra-tumoural injection with 2×10ˆ 7 pfu of DISC-GM-CSF, prepared as described above. Three hours after injection with DISC-GM-CSF, the mice were injected intra-tumourally with a preparation of 5×10ˆ 5 dendritic cells, obtained using the procedure described above. Two days after injection with dendritic cells, the mice were again injected intra-tumourally with 2×10ˆ 7 pfu of DISC-GM-CSF. Control groups of mice were injected intra-tumourally with either a) 2×10ˆ 7 pfu of DISC virus expressing GM-CSF alone, or b) a preparation of 5×10ˆ 5 dendritic cells, or c) 50 microliters of RPMI medium.

At days 0, 3, 6, 10, 13 and 50 after the final injection the surface area of the tumours was measured using calipers to give two perpendicular tumour measurements.

### Example 2

In this example of a treatment process according to the invention a mouse model system was used to test the efficacy of administration of a virus expressing murine GM-CSF followed by administration of a preparation of dendritic cells to treat tumours, as described in example 1, except that the treated mice were injected intra-tumourally with a preparation of 4×10ˆ 5 dendritic cells and mice in control group b) were injected with a preparation of 4×10ˆ 5 dendritic cells.

### Example 3

In this example of a treatment process according to the invention a mouse model system was used to test the efficacy of administration of a virus expressing murine GM-CSF followed by administration of a preparation of dendritic cells to treat tumours, as described in example 1, except that the treated mice were injected intra-tumourally with a preparation of 3×10ˆ 5 dendritic cells and mice in control group b) were injected with a preparation of 3×10ˆ 5 dendritic cells.

When any one of the tumours reached 10 mm in diameter the mouse was killed.

The reduction in the surface area of tumours in groups of mice treated with DISC-GM-CSF followed by dendritic cells according to examples 1-3 above, when compared to the control groups was a measure of the protection afforded by the combination therapy of DISC-GM-CSF, and by dendritic cells.

It was observed that treatment with DISC-GM-CSFand dendritic cells according to examples 1-3 above, can lead to increased tumour regression in comparison to treatment with DISC-GM-CSF or dendritic cells alone.

The present invention and disclosure extends to the methods and compositions and the resulting products as described herein, and to modifications and variations of the steps and features mentioned in the present description, including all combinations and subcombinations of the steps and features hereof, including variations in the order and selection of the steps, and the documents cited herein are hereby incorporated by reference in their entirety for all purposes.

We claim:

1. A method of treating target cells to damage them and/or reduce their proliferation wherein said method comprises the steps of (a) exposing the target cells to a cell-damaging agent, and also (b) exposing said target cells to a preparation of antigen-presenting cells, thereby to damage said cells and/or reduce their proliferation.

2. A method according to claim 1, wherein step (b) is carried out at least about 30 minutes after step (a).

3. A method according to claim 1, wherein the antigen-presenting cells consist essentially of dendritic cells.

4. A method according to claim 1, wherein the cell-damaging agent consists essentially of a virus vector for gene delivery.

5. A method according to claim 4, wherein the vector comprises one or more gene sequences encoding an immunomodulatory protein and/or a tumour antigen, or a functional fragment thereof.

6. A method according to claim 1, wherein the cell-damaging agent and the antigen-presenting cells are delivered to target cells in vivo

7. A method according to claim 1, wherein the cell-damaging agent and the antigen-presenting cells are delivered to target cells in vitro and the treated target cells are then implanted into a subject.

8. A method of treating cell proliferation in a subject which comprises administering to said subject separately or concurrently a cell-damaging agent and a preparation of antigen presenting cells in combination with a pharmaceutical excipient.
